SEQUENCE LISTING (26 sequences)

SEQ ID NO: 1 - DNA, 16 bp, Artificial Sequence
  Description of Combined DNA/RNA MoleculePNA Probe Sequence
ttcctccgttcgttcg

SEQ ID NO: 2 - DNA, 16 bp, Artificial Sequence
  Description of Combined DNA/RNA Molecule PNA Probe Sequence
taaggtcattcgttcg

SEQ ID NO: 3 - DNA, 17 bp, Artificial Sequence
  Description of Combined DNA/RNA Molecule PNA Probe Sequence
ttcgtctgttcgttcga

SEQ ID NO: 4 - DNA, 15 bp, Artificial Sequence
  Description of Combined DNA/RNA MoleculePNA Probe Sequence
aactttggaagagca

SEQ ID NO: 5 - DNA, 14 bp, Artificial Sequence
  Description of Combined DNA/RNA MoleculePNA Probe Sequence
agcaccaaaggagc

SEQ ID NO: 6 - DNA, 15 bp, Artificial Sequence
  Description of Combined DNA/RNA MoleculePNA Probe Sequence
ccccaacttacaggc

SEQ ID NO: 7 - DNA, 18 bp, Artificial Sequence
  Description of Combined DNA/RNA MoleculePNA Probe Sequence
actcttatccttgttctt

SEQ ID NO: 8 - DNA, 14 bp, Artificial Sequence
  Description of Combined DNA/RNA MoleculePNA Probe Sequence
aagggacaagcagt

SEQ ID NO: 9 - DNA, 17 bp, Artificial Sequence
  Description of Combined DNA/RNA MoleculePNA Probe Sequence
cactccagtcttccagt

SEQ ID NO: 10 - DNA, 17 bp, Artificial Sequence
  Description of Combined DNA/RNA MoleculePNA Probe Sequence
cactctaagtctccagt

SEQ ID NO: 11 - DNA, 15 bp, Artificial Sequence
  Description of Combined DNA/RNA MoleculePNA Probe Sequence
ggaaagctctgtctc

SEQ ID NO: 12 - DNA, 17 bp, Artificial Sequence
  Description of Combined DNA/RNA MoleculePNA Probe Sequence
ggttaccctaccgactt

SEQ ID NO: 13 - DNA, 17 bp, Artificial Sequence
  Description of Combined DNA/RNA MoleculePNA Probe Sequence
taaaggttaccctaccg

SEQ ID NO: 14 - DNA, 16 bp, Artificial Sequence
  Description of Combined DNA/RNA MoleculePNA Probe Sequence
gccacactttatcatt

SEQ ID NO: 15 - DNA, 16 bp, Artificial Sequence
  Description of Combined DNA/RNA MoleculePNA PRobe Sequence
gccacatcttatcatt

SEQ ID NO: 16 - DNA, 13 bp, Artificial Sequence
  Description of Combined DNA/RNA MoleculePNA Probe Sequencing
ttcaaaagcgtgg

SEQ ID NO: 17 - DNA, 13 bp, Artificial Sequence
  Description of Combined DNA/RNA MoleculePNA Probe Sequencing
ttcaaaggcgtgg

SEQ ID NO: 18 - DNA, 17 bp, Artificial Sequence
  Description of Combined DNA/RNA MoleculePNA Probe Sequencing
cctttgtactatccatt

SEQ ID NO: 19 - DNA, 17 bp, Artificial Sequence
  Description of Combined DNA/RNA MoleculePNA Probe Sequencing
gtactatccaatgtagc

SEQ ID NO: 20 - DNA, 17 bp, Artificial Sequence
  Description of Combined DNA/RNA MoleculePNA Probe Sequencing
gaccctttgtactattt

SEQ ID NO: 21 - DNA, 15 bp, Artificial Sequence
  Description of Combined DNA/RNA MoleculePNA Probe Sequencing
tgggattagctccac

SEQ ID NO: 22 - DNA, 15 bp, Artificial Sequence
  Description of Combined DNA/RNA MoleculePNA Probe Sequencing
gattagctccacctc

SEQ ID NO: 23 - DNA, 17 bp, Artificial Sequence
  Description of Combined DNA/RNA MoleculePNA Probe Sequencing
ctgagaatagttttatg

SEQ ID NO: 24 - DNA, 17 bp, Artificial Sequence
  Description of Combined DNA/RNA MoleculePNA Probe Sequencing
agaatagttttatggga

SEQ ID NO: 25 - DNA, 19 bp, Artificial Sequence
  Description of Combined DNA/RNA MoleculePNA Probe Sequencing
atagttttatgggattagc

SEQ ID NO: 26 - DNA, 17 bp, Artificial Sequence
  Description of Combined DNA/RNA MoleculePNA Probe Sequencing
taaattatctatgctaa

(19) United States
(12) Patent Application Publication
(10) Pub. No.: US 2004/0005611 A1
(43) Pub. Date: Jan. 8, 2004

(54) PNA probes, probe sets, methods and kits pertaining to the determination of Listeria

(76) Inventors: Jens J. Hyldig-Nielsen, Holliston, MA (US); Susan Rigby, Acton, MA (US); Byron F. Brehm-Stecher, Madison, WI (US); Eric A. Johnson, Madison, WI (US)

Correspondence Address: BRIAN D. GILDEA, APPLIED BIOSYSTEMS, 15 DEANGELO DRIVE, BEDFORD, MA, 01730, US

(21) Appl. No.: 10/436,962

(22) Filed: May 13, 2003

Related U.S. Application Data
(60) Provisional application No. 60/381,132, filed on May 17, 2002.

(51) Int. Cl. C12Q 1/68; C07K 14/00

(52) U.S. Cl. 435/6; 530/395

(57) ABSTRACT

This invention is related to novel PNA probes, probe sets, methods and kits pertaining to the determination of organisms of the Listeria genus and/or organisms of Listeria monocytogenes.

### CROSS REFERENCE TO RELATED APPLICATIONS

This application claims the benefit of U.S. Provisional Application No. 60/381,132 filed on May 17, 2002.

### BACKGROUND OF THE INVENTION

1. Field of the Invention

This invention is related to the field of probe-based detection, analysis and/or quantitation of microorganisms. More specifically, this invention relates to novel PNA probes, probe sets, methods and kits pertaining for the detection, identification and/or enumeration of organisms of the various species of the Listeria genus.

2. Description of the Related Art

Nucleic acid hybridization is a fundamental process in molecular biology. Probe-based assays are useful in the detection, quantitation and/or analysis of nucleic acids. Nucleic acid probes have long been used to analyze samples for the presence of nucleic acid from bacteria, fungi, virus or other organisms and are also useful in examining genetically-based disease states or clinical conditions of interest. Nonetheless, probe-based assays have been slow to achieve commercial success. This lack of commercial success is, at least partially, the result of difficulties associated with specificity, sensitivity and reliability.

Despite its name, Peptide Nucleic Acid (PNA) is neither a peptide, a nucleic acid nor is it an acid. Peptide Nucleic Acid (PNA) is a non-naturally occurring polyamide that can hybridize to nucleic acid (DNA and RNA) with sequence specificity (See: U.S. Pat. No. 5,539,082 and Egholm et al., Nature 365: 566-568 (1993)). Being a non-naturally occurring molecule, unmodified PNA is not known to be a substrate for the enzymes that are known to degrade peptides or nucleic acids. Therefore, PNA should be stable in biological samples, as well as have a long shelf-life. Unlike nucleic acid hybridization, which is very dependent on ionic strength, the hybridization of a PNA with a nucleic acid is fairly independent of ionic strength and is favored at low ionic strength, conditions that strongly disfavor the hybridization of nucleic acid to nucleic acid (Egholm et al., Nature, at p. 567). The effect of ionic strength on the stability and conformation of PNA complexes has been extensively investigated (Tomac et al., J. Am. Chem. Soc. 118:55 44-5552 (1996)). Sequence discrimination is more efficient for PNA recognizing DNA than for DNA recognizing DNA (Egholm et al., Nature, at p. 566). However, the advantages in point mutation discrimination with PNA probes, as compared with DNA probes, in a hybridization assay, appears to be somewhat sequence dependent (Nielsen et al., Anti-Cancer Drug Design 8:53-65, (1993) and Weiler et al., Nucl. Acids Res. 25: 2792-2799 (1997)).

Though they hybridize to nucleic acid with sequence specificity (See: Egholm et al., Nature, at p. 567), PNAs have been slow to achieve commercial success at least partially due to cost, sequence specific properties/problems associated with solubility and self-aggregation (See: Bergman, F., Bannwarth, W. and Tam, S., Tett. Lett. 36:6823-6826 (1995), Haaima, G., Lohse, A., Buchardt, O. and Nielsen, P. E., Angew. Chem. Int. Ed. Engl. 35:1939-1942 (1996) and Lesnik, E., Hassman, F., Barbeau, J., Teng, K. and Weiler, K., Nucleosides & Nucleotides 16:1775-1779 (1997) at p 433, col. 1, ln. 28 through col. 2, ln. 3) as well as the uncertainty pertaining to non-specific interactions that might occur in complex systems such as a cell (See: Good, L. et al., Antisense & Nucleic Acid Drug Development 7:431-437 (1997)). However, problems associated with solubility and self-aggregation have been reduced or eliminated (See: Gildea et al., Tett. Lett. 39: 7255-7258 (1998)). Nevertheless, their unique properties clearly demonstrate that PNA is not the equivalent of a nucleic acid in either structure or function. Consequently, PNA probes should be evaluated for performance and optimization to thereby confirm whether they can be used to specifically and reliably detect a particular nucleic acid target sequence, particularly when the target sequence exists in a complex sample such as a cell, tissue or organism.

### SUMMARY OF THE INVENTION

This invention is directed to PNA probes, probe sets, methods and kits useful for detecting, identifying and/or quantitating Listeria bacteria in a sample. The PNA probes, probe sets, methods and kits of this invention can be used for the analysis of nucleic acid, whether or not it is present within an organism of interest. Accordingly, this invention can be used for both the analysis of organisms or for the analysis of nucleic acid extracted from or derived from an organism of interest.

Generally, this invention can be useful for the determination of Listeria bacteria. The PNA probes and the probes of the probe sets of this invention comprise probing nucleobase sequences that are particularly useful for the specific detection of Listeria. In one embodiment, the probing nucleobase sequences are selected for determining organisms of the Listeria genus. In another embodiment, the probing nucleobase sequences are selected for determining Listeria monocytogenes. Exemplary probing nucleobase sequences for the probes of this invention are listed in Table 1, below. The Table identifies each sequence as being selected to determine either the Listeria genus or Listeria monocytogenes.

In one embodiment, a method for determining Listeria in a sample comprises contacting the sample with one or more PNA probes, wherein suitable probes are described herein. According to the method, the presence, absence and/or quantity of Listeria in the sample is then detected, identified and/or quantitated. Depending on the probing nucleobase sequence, the determination can be for organisms of the Listeria genus, or be for determination of Listeria monocytogenes. Detection, identification and/or quantitation is made possible by correlating the hybridization, under suitable hybridization conditions or suitable in-situ hybridization conditions, of the probing nucleobase sequence of a PNA probe or probes to the target sequence with the presence, absence and/or quantity of target organism in the sample. This correlation is made possible by direct or indirect determination of the probe/target sequence hybrid.

In yet another embodiment, this invention is directed to kits suitable for performing an assay that determines the presence, absence and/or quantity of Listeria in a sample. The kits of this invention comprise one or more PNA probes and other reagents, buffers or compositions that are selected to perform an assay or otherwise simplify the performance of an assay.

The PNA probes, probe sets, methods and kits of this invention have been demonstrated to be useful for organisms of the Listeria genus, or for Listeria monocytogenes, as the case may be. Moreover, the assays described herein are rapid (2-3 hours or less), sensitive, reliable and capable, in a single assay, of identification as well as detection and/or enumeration of the organisms listed in Table 1.

The PNA probes, probe sets, methods and kits of this invention can be particularly useful for the determination of Listeria in food, beverages, water, pharmaceutical products, personal care products, dairy products and/or environmental samples. The analysis of beverages includes soda, bottled water, fruit juice, beer, wine or liquor products. Suitable PNA probes, probe sets, methods and kits can be particularly useful for the analysis of raw materials, equipment, products or processes used to manufacture or store food, beverages, water, pharmaceutical products, personal care products dairy products or for the analysis of environmental samples.

Additionally, the PNA probes, probe sets, methods and kits of this invention can be particularly useful for the detection of Listeria species in clinical samples and clinical environments. Non-limiting examples of clinical samples include: sputum, laryngeal swabs, gastric lavage, bronchial washings, biopsies, aspirates, expectorates, body fluids (e.g. spinal, pleural, pericardial, synovial, blood, pus, amniotic, and urine), bone marrow and tissue sections (including cultures and subcultures derived therefrom). Suitable PNA probes, probe sets, methods and kits will also be particularly useful for the analysis of clinical specimens, equipment, fixtures or products used to treat humans or animals.

### DETAILED DESCRIPTION OF THE INVENTION

1. Definitions:

a. As used herein, “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobase include those nucleobases illustrated in FIGS. 2(A) and 2(B) of Buchardt et al. of U.S. Pat. No. 6,357,163 (incorporated herein by reference).

b. As used herein, “nucleobase sequence” means any segment, or aggregate of two or more segments of a polymer that comprises nucleobase-containing subunits. Non-limiting examples of suitable polymers include oligodeoxynucleotides (e.g. DNA), oligoribonucleotides (e.g. RNA), peptide nucleic acids (PNA), PNA chimeras, PNA oligomers, nucleic acid analogs and/or nucleic acid mimics.

c. As used herein, “target sequence” is a nucleobase sequence of a polynucleobase strand sought to be determined. The target sequence can be a subsequence of the rRNA of Listeria bacteria. The target sequence can also be a subsequence of cDNA or cRNA of the rRNA of Listeria.

d. As used herein, “polynucleobase strand” means a complete single polymer strand comprising nucleobase subunits.

e. As used herein, “nucleic acid” is a nucleobase sequence-containing polymer, or polynucleobase strand, having a backbone formed from nucleotides, or analogs thereof. Preferred nucleic acids are DNA and RNA. For the avoidance of any doubt, PNA is a nucleic acid mimic and not a nucleic acid analog.

f. As used herein, “peptide nucleic acid” or “PNA” means any oligomer or polymer segment comprising two or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 and 6,357,163; all of which are herein incorporated by reference. The term “peptide nucleic acid” or “PNA” shall also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 11:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

In certain embodiments, a “peptide nucleic acid” or “PNA” is an oligomer or polymer segment comprising two or more covalently linked subunits of the formula:

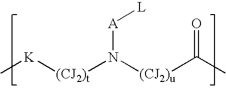

wherein, each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms that may optionally contain a heteroatom or a substituted or unsubstituted aryl group. Each A is selected from the group consisting of a single bond, a group of the formula; —(CJ₂)ₛ— and a group of the formula; —(CJ₂)ₛC(O)—, wherein, J is defined above and each s is a whole number from one to five. Each t is 1 or 2 and each u is 1 or 2. Each L is the same or different and is independently selected from: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6diaminopurine), hypoxanthine, N9-(7-deaza-guanine), N9-(7-deaza-8-aza-guanine) and N8-(7-deaza-8-aza-adenine), other naturally occurring nucleobase analogs or other non-naturally occurring nucleobases.

In certain other embodiments, a PNA subunit consists of a naturally occurring or non-naturally occurring nucleobase attached to the N-α-glycine nitrogen of the N-[2-(aminoethyl)]glycine backbone through a methylene carbonyl linkage; this currently being the most commonly used form of a peptide nucleic acid subunit.

g. As used herein, the terms “label”, “reporter moiety” or “detectable moiety” are interchangeable and refer to moieties that can be attached to PNA oligomer or antibody, or otherwise be used in a reporter system, to thereby render the oligomer or antibody detectable by an instrument or method. For example, a label can be any moiety that: (i) provides a detectable signal; (ii) interacts with a second label to modify the detectable signal provided by the first or second label; or (iii) confers a capture function, i.e. hydrophobic affinity, antibody/antigen, ionic complexation.

h. As used herein, “sequence specifically” means hybridization by base pairing through hydrogen bonding. Non-limiting examples of standard base pairing includes adenine base pairing with thymine or uracil and guanine base pairing with cytosine. Other non-limiting examples of base-pairing motifs include, but are not limited to: adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); thymine or uracil base pairing with any of: 2-aminopurine, N9-(2-amino-6-chloropurine) or N9-(2,6-diaminopurine); and N8-(7-deaza-8-aza-adenine), being a universal base, base pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine) or N9-(7-deaza-8-aza-guanine) (See: Seela et al., Nucl. Acids, Res.: 28(17): 3224-3232 (2000)).

i. As used herein, the term “chimera” or “chimeric oligomer” means an oligomer comprising two or more linked subunits that are selected from different classes of subunits. For example, a PNA/DNA chimera would comprise at least two PNA subunits linked to at least one 2′-deoxyribonucleic acid subunit (For exemplary methods and compositions related to PNA/DNA chimera preparation See: WO96/40709). Exemplary component subunits of the chimera are selected from the group consisting of PNA subunits, naturally occurring amino acid subunits, DNA subunits, RNA subunits and subunits of analogues or mimics of nucleic acids.

j. As used herein, the term “linked polymer” means a polymer comprising two or more polymer segments which are linked by a linker. The polymer segments that can be linked to form the linked polymer can be selected from the group consisting of an oligodeoxynucleotide, an oligoribonucleotide, a peptide, a polyamide, a peptide nucleic acid (PNA) and a chimera.

k. As used herein “solid support” or “solid carrier” means any solid phase material upon which a oligomer is synthesized, attached, ligated or otherwise immobilized. Solid support encompasses terms such as “resin”, “solid phase”, “surface” and “support”. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

1. As used herein, “support bound” means immobilized on or to a solid support. It is understood that immobilization can occur by any means, including for example; by covalent attachment, by electrostatic immobilization, by attachment through a ligand/ligand interaction, by contact or by depositing on the surface.

2. Description

I. General:

PNA Synthesis:

Methods for the chemical assembly of PNAs are well known (See: Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 and 6,107,470; all of which are herein incorporated by reference (Also see: PerSeptive Biosystems Product Literature)). As a general reference for PNA synthesis methodology also please see: Nielsen et al., Peptide Nucleic Acids; Protocols and Applications, Horizon Scientific Press, Norfolk England (1999).

Chemicals and instrumentation for the support bound automated chemical assembly of peptide nucleic acids are now commercially available. Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkyl amino terminus that can be condensed with the next synthon to be added to the growing polymer. Because standard peptide chemistry is utilized, natural and non-natural amino acids can be routinely incorporated into a PNA oligomer. Because a PNA is a polyamide, it has a C-terminus (carboxyl terminus) and an N-terminus (amino terminus). For the purposes of the design of a hybridization probe suitable for antiparallel binding to the target sequence (the preferred orientation), the N-terminus of the probing nucleobase sequence of the PNA probe is the equivalent of the 5′-hydroxyl terminus of an equivalent DNA or RNA oligonucleotide.

PNA Labeling:

Non-limiting methods for labeling PNAs are described in U.S. Pat. Nos. 6,110,676, 6,361,942, 6,355,421 (all incorporated herein by reference), WO99/21881, the examples section of this specification or are otherwise well known in the art of PNA synthesis. Other non-limiting examples for labeling PNAs are also discussed in Nielsen et al., Peptide Nucleic Acids; Protocols and Applications, Horizon Scientific Press, Norfolk England (1999).

Labels:

Non-limiting examples of detectable moieties (labels) that can be used to label PNA probes or antibodies used in the practice of this invention can include a dextran conjugate, a branched nucleic acid detection system, a chromophore, a fluorophore, a spin label, a radioisotope, an enzyme, a hapten, an acridinium ester or a chemiluminescent compound. Other suitable labeling reagents and preferred methods of attachment would be recognized by those of ordinary skill in the art of PNA, peptide or nucleic acid synthesis.

Non-limiting examples of haptens include 5(6)-carboxyfluorescein, 2,4-dinitrophenyl, digoxigenin, and biotin.

Non-limiting examples of fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.) or the Alexa dye series (Molecular Probes, Eugene, Oreg.).

Non-limiting examples of enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP), soy bean peroxidase (SBP)), ribonuclease and protease.

Energy Transfer

In one embodiment, PNA oligomers can be labeled with an energy transfer set. For energy transfer to be useful in determining hybridization, there should be an energy transfer set comprising at least one energy transfer donor and at least one energy transfer acceptor moiety. Often, the energy transfer set will include a single donor moiety and a single acceptor moiety, but this is not a limitation. An energy transfer set may contain more than one donor moiety and/or more than one acceptor moiety. The donor and acceptor moieties operate such that one or more acceptor moieties accept energy transferred from the one or more donor moieties or otherwise quench the signal from the donor moiety or moieties. Thus, in one embodiment, both the donor moiety(ies) and acceptor moiety(ies) are fluorophores. Though the previously listed fluorophores (with suitable spectral properties) might also operate as energy transfer acceptors, the acceptor moiety can also be a non-fluorescent quencher moiety such as 4-((-4-(dimethylamino)phenyl)azo) benzoic acid (dabcyl). The labels of the energy transfer set can be linked at the oligomer termini or linked at a site within the oligomer. For example, each of two labels of an energy transfer set can be linked at the distal-most termini of the oligomer.

Transfer of energy between donor and acceptor moieties may occur through any energy transfer process, such as through the collision of the closely associated moieties of an energy transfer set(s) or through a non-radiative process such as fluorescence resonance energy transfer (FRET). For FRET to occur, transfer of energy between donor and acceptor moieties of a energy transfer set requires that the moieties be close in space and that the emission spectrum of a donor(s) have substantial overlap with the absorption spectrum of the acceptor(s) (See: Yaron et al. Analytical Biochemistry, 95: 228-235 (1979) and particularly page 232, col. 1 through page 234, col. 1). Alternatively, collision mediated (radiationless) energy transfer may occur between very closely associated donor and acceptor moieties whether or not the emission spectrum of a donor moiety(ies) has a substantial overlap with the absorption spectrum of the acceptor moiety(ies) (See: Yaron et al., Analytical Biochemistry, 95: 228-235 (1979) and particularly page 229, col. 1 through page 232, col. 1). This process is referred to as intramolecular collision since it is believed that quenching is caused by the direct contact of the donor and acceptor moieties (See: Yaron et al.). Energy transfer can also occur through processes for which the mechanism of action has yet to be described. It is to be understood that any reference to energy transfer in the instant application encompasses all of these mechanistically distinct phenomena. It is also to be understood that energy transfer can occur though more than one energy transfer process simultaneously and that the change in detectable signal can be a measure of the activity of two or more energy transfer processes. Accordingly, the mechanism of energy transfer is not a limitation of this invention.

Detecting Energy Transfer in a Self-Indicating PNA Oligomer:

When labeled with an energy transfer set, we refer to the PNA oligomer as being self-indicating. In one embodiment, a self-indicating PNA oligomer can be labeled in a manner that is described in co-pending and commonly owned patent application U.S. Ser. No. 09/179,162 (now allowed), entitled: “Methods, Kits And Compositions Pertaining To Linear Beacons” and the related PCT application which has also now published as WO99/21881, both of which are hereby incorporated by reference.

Hybrid formation between a self-indicating oligomer and a target sequence can be monitored by measuring at least one physical property of at least one member of the energy transfer set that is detectably different when the hybridization complex is formed as compared with when the oligomer exists in a non-hybridized state. We refer to this phenomenon as the self-indicating property of the oligomer. This change in detectable signal results from the change in efficiency of energy transfer between donor and acceptor moieties caused by hybridization of the oligomer to the target sequence.

For example, the means of detection can involve measuring fluorescence of a donor or acceptor fluorophore of an energy transfer set. In one embodiment, the energy transfer set may comprise at least one donor fluorophore and at least one acceptor (fluorescent or non-fluorescent) quencher such that the measure of fluorescence of the donor fluorophore can be used to detect, identify or quantitate hybridization of the oligomer to the target sequence. For example, there may be a measurable increase in fluorescence of the donor fluorophore upon the hybridization of the oligomer to a target sequence.

In another embodiment, the energy transfer set comprises at least one donor fluorophore and at least one acceptor fluorophore such that the measure of fluorescence of either, or both, of at least one donor moiety or one acceptor moiety can be used to can be used to detect, identify or quantitate hybridization of the oligomer to the target sequence.

Self-indicating PNA oligomers can be used in in-situ hybridization assays. However, certain self-indicating PNA oligomers are particularly well suited for the analysis of nucleic acid amplification reactions (e.g. PCR) either in real-time or at the end point (See: WO99/21881).

Determining Energy Transfer in a Detection Complex:

In another embodiment, the PNA oligomers of the present invention are labeled solely with a quencher moiety and can be used as a component oligomer in a Detection Complex as more fully explained in U.S. Pat. No. 6,361,942, entitled: “Methods, Kits And Compositions Pertaining To Detection Complexes”, herein incorporated by reference. When the Detection Complex is formed, at least one donor moiety of one component polymer is brought sufficiently close in space to at least one acceptor moiety of a second component polymer. Since the donor and acceptor moieties of the set are closely situated in space, transfer of energy occurs between moieties of the energy transfer set. When the Detection Complex dissociates, as for example when one of the component polymers of the Detection Complex hybridizes to a target sequence, the donor and acceptor moieties do not interact sufficiently to cause substantial transfer of energy from the donor and acceptor moieties of the energy transfer set and there is a correlating change in detectable signal from the donor and/or acceptor moieties of the energy transfer set. Consequently, Detection Complex formation/dissociation can be determined by measuring at least one physical property of at least one member of the energy transfer set that is detectably different when the complex is formed as compared with when the component polymers of the Detection Complex exist independently and unassociated.

Detectable and Independently Detectable Moieties/Multiplex Analysis:

A multiplex hybridization assay can be performed in accordance with this invention. In a multiplex assay, numerous conditions of interest can be simultaneously examined. Multiplex analysis relies on the ability to sort sample components or the data associated therewith, during or after the assay is completed. In preferred embodiments of the invention, one or more distinct independently detectable moieties can be used to label two or more different probes used in an assay. The ability to differentiate between and/or quantitate each of the independently detectable moieties provides the means to multiplex a hybridization assay because the data that correlates with the hybridization of each of the distinctly (independently) labeled probe to a particular nucleic acid sequence can be correlated with the presence, absence or quantity of each organism sought to be detected in the sample. Consequently, the multiplex assays of this invention can be used to simultaneously detect the presence, absence or quantity of two or more different organisms (e.g. species of Listeria) in the same sample and in the same assay. For example, a multiplex assay may utilize two or more PNA probes, each being labeled with an independently detectable fluorophore, or a set of independently detectable fluorophores.

Spacer/Linker Moieties:

Generally, spacers are used to minimize the adverse effects that bulky labeling reagents might have on hybridization properties of probes. Linkers typically induce flexibility and randomness into the probe or otherwise link two or more nucleobase sequences of a probe or component polymer. Preferred spacer/linker moieties for the nucleobase polymers of this invention consist of one or more aminoalkyl carboxylic acids (e.g. aminocaproic acid) the side chain of an amino acid (e.g. the side chain of lysine or ornithine), natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane). Spacer/linker moieties may also incidentally or intentionally be constructed to improve the water solubility of the probe (For example see: Gildea et al., Tett. Lett. 39: 7255-7258 (1998)).

For example, a spacer/linker moiety can comprise one or more linked compounds having the formula: —Y—(Oₘ—(CW₂)ₙ)ₒ-Z-. The group Y is selected from the group consisting of: a single bond, —(CW₂)ₚ—,—C(O)(CW₂)ₚ—, —C(S)(CW₂)ₚ— and —S(O₂)(CW₂)ₚ. The group Z has the formula NH, NR², S or O. Each W is independently H, R², —OR², F, Cl, Br or I; wherein, each R² is independently selected from the group consisting of: —CX₃, —CX₂CX₃, —CX₂CX₂CX₃, —CX₂CX(CX₃)₂, and —C(CX₃)₃. Each X is independently H, F, Cl, Br or I. Each m is independently 0 or 1. Each n, o and p are independently integers from 0 to 10.

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target combination can often be found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

Suitable Hybridization Conditions:

Generally, the more closely related the background causing nucleic acid contaminates are to the target sequence, the more careful stringency must be controlled. Blocking probes may also be used as a means to improve discrimination beyond the limits possible by mere optimization of stringency factors. Suitable hybridization conditions will thus comprise conditions under which the desired degree of discrimination is achieved such that an assay generates an accurate (within the tolerance desired for the assay) and reproducible result. Aided by no more than routine experimentation and the disclosure provided herein, those of skill in the art will easily be able to determine suitable hybridization conditions for performing assays utilizing the methods, kits and compositions described herein. Suitable in-situ hybridization conditions comprise conditions suitable for performing an in-situ hybridization procedure. Thus, suitable hybridization or suitable in-situ hybridization conditions will become apparent using the disclosure provided herein; with or without additional routine experimentation.

Blocking Probes:

Blocking probes are nucleic acid or non-nucleic acid probes (e.g. PNA probes) that can be used to suppress the binding of the probing nucleobase sequence of the probing polymer to a non-target sequence. Preferred blocking probes are PNA probes (See: Coull et al., WIPO publication No. WO98/24933 as well as U.S. Pat. No. 6,110,676). Typically, blocking probes are closely related to the probing nucleobase sequence and preferably they comprise a point mutation as compared with the probing nucleobase sequence. It is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence. Thus, blocking probes can be used with the methods, kits and compositions of this invention to suppress the binding of the PNA probe to a non-target sequence that might be present and interfere with the performance of the assay. Blocking probes are particularly advantageous in single point mutation discrimination. Non-limiting examples of blocking probes that can be used in assays for the determination Listeria monocytogenes can be found in Table 1.

Probing Nucleobase Sequence:

The probing nucleobase sequence of a PNA probe is the specific sequence recognition portion of the construct. Therefore, the probing nucleobase sequence is a sequence of PNA subunits designed to sequence specifically hybridize to a target sequence wherein the presence, absence and/or amount of target sequence can be used to detect the presence, absence and/or quantity of Listeria in a sample. Consequently, with due consideration of the requirements of a PNA probe for the assay format chosen, the length of the probing nucleobase sequence of the PNA probe will generally be chosen such that a stable complex is formed with the target sequence under suitable hybridization conditions or suitable in-situ hybridization conditions.

The probing nucleobase sequence suitable for detecting the target organism listed in Seq. Id. Nos. 1-31 of Table 1, will generally, but not necessarily, have a length of 18 or fewer PNA subunits wherein the exact nucleobase sequence can be at least 90% homologous to the probing nucleobase sequences listed in Table 1, or their complements. The PNA probes can be 100% homologous to said sequences or can comprise the exact nucleobase sequences appearing the Table 1. The probing nucleobase sequence can be exactly identical to those nucleobase sequences listed in Table 1. Complements of the probing nucleobase sequences listed in Seq. Id. Nos. 1-31 of Table 1 are included since it is possible to prepare or amplify copies of the target sequence wherein the copies are complements of the target sequence and thus, will bind to the complement of the probing nucleobase sequences listed in Table 1. Useful probing nucleobase sequences are listed in Table 1. These probing nucleobase sequences have been shown to be specific for the determination of organisms of the genus of Listeria or for Listeria monocytogenes, as the case may be (See information listed in Table 1 and the Examples, below).

A PNA probe of this invention will generally have a probing nucleobase sequence that is complementary to the target sequence. Alternatively, a substantially complementary probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15:331-335 (1997)).

This invention contemplates that variations in the probing nucleobase sequences listed in Table 1 shall provide PNA probes that are suitable for the specific detection of the organisms listed. Common variations include, deletions, insertions and frame shifts. Variation of the probing nucleobase sequences within the parameters described herein are considered to be an embodiment of this invention.

Probe Complexes:

In still another embodiment, two probes are designed to hybridize to the target sequence sought to be detected to thereby generate a detectable signal whereby the probing nucleobase sequence of each probe comprises half or approximately half of the nucleobase sequence required for hybridization to the complete target sequence of the organism sought to be detected in the assay such that the aggregate nucleobase sequence of the two probes forms the probing nucleobase sequence that hybridizes to the target sequence. As a non-limiting example, the probing nucleobase sequences of the two probes might be designed using the assay as described in U.S. Pat. No. 6,027,893, entitled: “Method of identifying a nucleic acid using triple helix formation of adjacently annealed probes” by H. Orum et al., herein incorporated by reference. Using this methodology, the probes that hybridize to the target sequence may or may not be labeled. However, it is the probe complex formed by the annealing of the adjacent probes that is detected. Similar compositions comprised solely of PNA probes have been described in U.S. Pat. No. 6,287,772, herein incorporated by reference.

II. Preferred Embodiments of the Invention:

a. PNA Probes:

In one embodiment, this invention is directed to PNA probes. The PNA probes of this invention are suitable for the determination of Listeria in a sample. For example, determination can be the detecting, identifying and/or quantitating of Listeria in a sample. The PNA probes, probe sets, methods and kits of this invention are suitable for the analysis of nucleic acid, whether or not it is present within an organism of interest. Accordingly, this invention can be used for both the analysis of organisms or for the analysis of nucleic acid extracted from or derived from an organism of interest. Thus, the source of the target sequence is not a limitation of this invention.

Generally, this invention can be useful for the determination of Listeria bacteria. The PNA probes and the probes of the probe sets of this invention comprise probing nucleobase sequences that are particularly useful for the specific detection of Listeria. In one embodiment, the probing nucleobase sequences are selected for determining organisms of the Listeria genus. This includes Seq. Id. Nos. 1-13. Seq. Id. Nos. 6 and 8 are particularly useful in this regard. Seq. Id. No. 8. is very useful since it is the only probing nucleobase sequence known by applicants that is capable of determining all known species of Listeria. In another embodiment, the probing nucleobase sequences are selected for determining Listeria monocytogenes. This includes Seq. Id. Nos. 14-31. Seq. Id. Nos. 5, 8, 9 and 10 are particularly useful in this regard. General characteristics (e.g. length, labels, linkers etc.) of PNA probes suitable for determining these bacteria have been previously described herein. Exemplary probing nucleobase sequences for the probes of this invention are listed in Table 1, below. Table 1 identifies whether each nucleobase sequence as being selected to determine organisms of the Listeria genus or Listeria monocytogenes.

The PNA probes of this invention may comprise only a probing nucleobase sequence (as previously described herein) or may comprise additional moieties. Non-limiting examples of additional moieties include detectable moieties (labels), linkers, spacers, natural or non-natural amino acids, peptides, enzymes and/or other subunits of PNA, DNA or RNA. Additional moieties may be functional or non-functional in an assay. Generally however, additional moieties will be selected to be functional within the design of the assay in which the PNA probe is to be used. For example, the PNA probes of this invention can be labeled with one or more detectable moieties or labeled with two or more independently detectable moieties. The independently detectable moieties can be independently detectable fluorophores.

TABLE 1

| | | |
|---|---|---|
| Seq. | | |
| ID. | Target | |
| No. | Organism | Probing Nucleobase Sequence |

| | | |
|---|---|---|
| 1 | Listeria | TTC-CTC-CGT-TCG-TTC-G |
| 2 | Listeria | TAA-GGT-CAT-TCG-TTC-G |
| 3 | Listeria | TTC-GTC-TGT-TCG-TTC-GA |
| 4 | Listeria | AAC-TTT-GGA-AGA-GCA |
| 5 | Listeria | ACG-ACC-AAA-GGA-GC |
| 6 | Listeria | CCC-CAA-CTT-ACA-GGC |
| 7 | Listeria | ACT-CTT-ATC-CTT-GTT-CTT |
| 8 | Listeria | AAG-GGA-CAA-GCA-GT |
| 9 | Listeria | CAC-TCC-AGT-CTT-CCA-GT |
| 10 | Listeria | CAC-TCT-AAG-TCT-CC-AGT |
| 11 | Listeria | GGA-AAG-CTC-TGT-CTC |
| 12 | Listeria | GGT-TAC-CCT-ACC-GAC-TT |
| 13 | Listeria | TAA-AGG-TTA-CCC-TAC-CG |
| 14 | L. monocytogenes | GCC-ACA-CTT-TAT-CAT-T |
| 15 | L. monocytogenes | GCC-ACA-TCT-TAT-CAT-T |
| 16 | L. monocytogenes | TTC-AAA-AGC-GTG-G |
| 17 | L. monocytogenes | TTC-AAA-GGC-GTG-G |
| 18 | L. monocytogenes | CCT-TTG-TAC-TAT-CCA-TT |
| 19 | L. monocytogenes | GTA-CTA-TCC-AAT-GTA-GC |
| 20 | L. monocytogenes | GAC-CCT-TTG-TAC-TAT-CC |
| 21 | L. monocytogenes | TGG-GAT-TAG-CTC-CAC |
| 22 | L. monocytogenes | GAT-TAG-CTC-CAC-CTC |
| 23 | L. monocytogenes | CTG-AGA-ATA-GTT-TTA-TG |
| 24 | L. monocytogenes | AGA-ATA-GTT-TTA-TGG-GA |
| 25 | L. monocytogenes | ATA-GTT-TTA-TGG-GAT-TAG-C |
| 26 | L. monocytogenes | TAA-ATT-ATC-TAT-GCT-AA |
| 27 | L. monocytogenes | TTC-TGA-TTT-TCC-GTA-TC |
| 28 | L. monocytogenes | GGT-TCC-CCC-ATT-CG |
| 29 | L. monocytogenes | AAA-GCC-ATT-TCA-ACT-A |
| 30 | L. monocytogenes | TAC-TTA-TGC-GCC-CTA |
| 31 | L. monocytogenes | ACG-AAC-CTC-TAA-AGA |
| 32 | Blocker Probe | CCT-TTG-TAC-CAT-CCA-TT |
| 33 | Blocker Probe | CCT-TTG-TAT-TAT-CCA-TT |
| 34 | Blocker Probe | CTG-AGA-ATG-GTT-TTA-TG |
| 35 | Blocker Probe | CTG-AGA-ATA-ATT-TTA-TG |

The probes of this invention can be used in in-situ hybridization (ISH) and fluorescence in-situ hybridization (FISH) assays. Excess probe used in an ISH or FISH assay often will be removed so that the detectable moiety of specifically bound probes can be detected above the background signal that results from still present but unhybridized probe. Generally, the excess probe can be washed away after the sample has been incubated with probe for a period of time. However, because certain types of self-indicating probes can generate little or no detectable background, they can be used to eliminate the requirement that excess probe be completely removed (washed away) from the sample.

Unlabeled Non-Nucleic Acid Probes:

The probes of this invention need not be labeled with a detectable moiety to be operable within the scope of this invention. When using the probes of this invention it is possible to detect the probe/target sequence complex formed by hybridization of the probing nucleobase sequence of the probe to the target sequence. For example, a PNA/nucleic acid complex formed by the hybridization of a PNA probing nucleobase sequence to the target sequence could be detected using an antibody that specifically interacts with the complex under antibody binding conditions. Suitable antibodies to PNA/nucleic acid complexes and methods for their preparation and use are described in WIPO Patent Application WO95/17430 and U.S. Pat. No. 5,612,458, herein incorporated by reference.

The antibody/PNA/nucleic acid complex formed by interaction of the α-PNA/nucleic acid antibody with the PNA/nucleic acid complex can be detected by several methods. For example, the α-PNA/nucleic acid antibody could be labeled with a detectable moiety. Suitable detectable moieties have been previously described herein. Thus, the presence, absence and/or quantity of the detectable moiety can be correlated with the presence, absence and/or quantity of the antibody/PNA/nucleic acid complex and the bacteria to be identified by the probing nucleobase sequence of the PNA probe. Alternatively, the antibody/PNA/nucleic acid complex can be detected using a secondary antibody that is labeled with a detectable moiety. Typically the secondary antibody specifically binds to the α-PNA/nucleic acid antibody under antibody binding conditions. Thus, the presence, absence and/or quantity of the detectable moiety can be correlated with the presence, absence and/or quantity of the antibody/antibody/PNA/nucleic acid complex and the bacteria to be identified by the probing nucleobase sequence of the probe. As used herein, the term antibody includes antibody fragments that specifically bind to other antibodies or other antibody fragments.

Immobilization of Probes to a Surface:

One or more of the PNA probes of this invention may optionally be immobilized to a surface for the detection of the target sequence of a target organism of interest. PNA probes can be immobilized to the surface using the well known process of UV-crosslinking. A PNA probe can be synthesized on the surface in a manner suitable for deprotection but not cleavage from the synthesis support (See: Weiler, J. et al, Hybridization based DNA screening on peptide nucleic acid (PNA) oligomer arrays., Nucl. Acids Res., 25, 14:2792-2799 (July 1997)). In still another embodiment, PNA probes can be covalently linked to a surface by the reaction of a suitable functional group on the probe with a functional group of the surface (See: Lester, A. et al, “PNA Array Technology”: Presented at Biochip Technologies Conference in Annapolis (October 1997)). This method is most advantageous since the PNA probes on the surface will typically be highly purified and attached using a defined chemistry, thereby minimizing or eliminating non-specific interactions.

Methods for the chemical attachment of probes to surfaces generally involve the reaction of a nucleophilic group, (e.g. an amine or thiol) of the probe to be immobilized, with an electrophilic group on the support to be modified. Alternatively, the nucleophile can be present on the support and the electrophile (e.g. activated carboxylic acid) present on the probe. Because native PNA possesses an amino terminus, a PNA will not necessarily require modification to thereby immobilize it to a surface (See: Lester et al., Poster entitled “PNA Array Technology”).

Conditions suitable for the immobilization of a PNA probe to a surface will generally be similar to those conditions suitable for the labeling of the polymer. The immobilization reaction is essentially the equivalent of labeling whereby the label is substituted with the surface to which the polymer is to be linked.

Numerous types of surfaces derivatized with amino groups, carboxylic acid groups, isocyantes, isothiocyanates and malimide groups are commercially available. Non-limiting examples of suitable surfaces include membranes, chips (e.g. silicone chips), glass, controlled pore glass, polystyrene particles (beads), silica and gold nanoparticles.

Arrays of PNA Probes or Probe Sets:

Arrays are surfaces to which two or more probes have been immobilized each at a specified position. The probing nucleobase sequence of the immobilized probes can be judiciously chosen to interrogate a sample that may contain nucleic acid from one or more target organisms. Because the location and composition of each immobilized probe is known, arrays can be useful for the simultaneous detection, identification and/or quantitation of nucleic acid from two or more target organisms that may be present in the sample. Moreover, arrays of PNA probes can be regenerated by stripping away any of the hybridized nucleic acid after each assay, thereby providing a means to repetitively analyze numerous samples using the same array. Thus, arrays of PNA probes or PNA probe sets may be useful for repetitive screening of samples for target organisms of interest. The arrays of this invention comprise at least one PNA probe (as described herein) suitable for the detection, identification and/or quantitation of at least one organism representing a genus or species of Listeria. Exemplary probing nucleobase sequences for the immobilized PNA probes are listed in Table 1.

b. PNA Probe Sets:

In another embodiment, this invention is directed to probe sets suitable for determining Listeria in a sample of interest. In one embodiment, the probe set comprises probes suitable for determining one or more organisms of the Listeria genus as well as one or more organisms of Listeria monocytogenes. In another embodiment, the probe set comprises at least one probe for determining organisms of the Listeria genus. In still another embodiment, the probe set comprises at least one probe for determining organisms of Listeria monocytogenes.

The general and preferred characteristics of PNA probes suitable for the determination of these bacteria have been previously described herein. Preferred probing nucleobase sequences for the target species are listed in Table 1. The grouping of PNA probes within sets characterized for specific types or groups of bacteria can be a very useful embodiment of this invention. The PNA probes of this invention can be combined with probes for other bacteria or even for organisms other than bacteria such as been described in U.S. Pat. No. 6,280,946, herein incorporated by reference, wherein a multiplex assay for both bacteria and yeast has been described using a single PNA probe set.

Probe sets of this invention comprise at least one PNA probe but need not comprise only PNA probes. For example, probe sets of this invention may comprise mixtures of PNA probes and nucleic acid probes, provided however that a set comprises at least one PNA probe as described herein. In one embodiment, some of the probes of the set can be blocking probes composed of PNA or nucleic acid. Non-limiting examples of nucleobase sequences suitable for use as blocking probes can be found in Table 1. In other embodiments, the probe set can be used to determine organisms other than Listeria in addition to the determination of at least one Listeria bacteria.

Table 1 lists two or more probing nucleobase sequences for the determination of organisms of either the Listeria genus or for Listeria monocytogenes. Where alternative probing nucleobase sequences exist, it can be advantageous to use a probe set containing the two or more PNA probes to thereby increase the detectable signal in the assay for either or both of organisms of the Listeria genus or of Listeria monocytogenes.

One exemplary probe set would comprise probes suitable for determining Listeria wherein two or more of the probes of the set comprise a probing nucleobase sequence selected from the group consisting of: TTC-CTC-CGT-TCG-TTC-G (Seq. Id. No. 1), TAA-GGT-CAT-TCG-TTC-G (Seq. Id. No. 2), TTC-GTC-TGT-TCG-TTC-GA (Seq. Id. No. 3), AAC-TTT-GGA-AGA-GCA (Seq. Id. No. 4), ACG-ACC-AAA-GGA-GC (Seq. Id. No. 5), CCC-CAA-CTT-ACA-GGC (Seq. Id. No. 6), ACT-CTT-ATC-CTT-GTT-CTT (Seq. Id. No. 7), AAG-GGA-CAA-GCA-GT (Seq. Id. No. 8), CAC-TCC-AGT-CTT-CCA-GT (Seq. Id. No. 9), CAC-TCT-AAG-TCT-CC-AGT (Seq. Id. No. 10), GGA-AAG-CTC-TGT-CTC (Seq. Id. No. 11), GGT-TAC-CCT-ACC-GAC-TT (Seq. Id. No. 12) and TAA-AGG-TTA-CCC-TAC-CG (Seq. Id. No. 13). A second exemplary probe set can comprise probes suitable for determining Listeria monocytogenes wherein the probes of the set comprise a probing nucleobase sequence selected from the group consisting of: GCC-ACA-CTT-TAT-CAT-T (Seq. Id. No. 14), GCC-ACA-TCT-TAT-CAT-T (Seq. Id. No. 15), TTC-AAA-AGC-GTG-G (Seq. Id. No. 16), TTC-AAA-GGC-GTG-G (Seq. Id. No. 17), CCT-TTG-TAC-TAT-CCA-TT (Seq. Id. No. 18), GTA-CTA-TCC-AAT-GTA-GC (Seq. Id. No. 19), GAC-CCT-TTG-TAC-TAT-CC (Seq. Id. No. 20), TGG-GAT-TAG-CTC-CAC (Seq. Id. No. 21), GAT-TAG-CTC-CAC-CTC (Seq. Id. No. 22), CTG-AGA-ATA-GTT-TTA-TG (Seq. Id. No. 23), AGA-ATA-GTT-TTA-TGG-GA (Seq. Id. No. 24), ATA-GTT-TTA-TGG-GAT-TAG-C (Seq. Id. No. 25) and TAA-ATT-ATC-TAT-GCT-AA (Seq. Id. No. 26). Still a third exemplary probe set can comprise probes suitable for determining both organisms of the Listeria genus as well as organisms of Listeria monocytogenes wherein at least one of the probes of the set comprises a probing nucleobase sequence selected from the group consisting of: TTC-CTC-CGT-TCG-TTC-G (Seq. Id. No. 1), TAA-GGT-CAT-TCG-TTC-G (Seq. Id. No. 2), TTC-GTC-TGT-TCG-TTC-GA (Seq. Id. No. 3), AAC-TTT-GGA-AGA-GCA (Seq. Id. No. 4), ACG-ACC-AAA-GGA-GC (Seq. Id. No. 5), CCC -CAA-CTT-ACA-GGC (Seq. Id. No. 6), ACT-CTT-ATC-CTT-GTT-CTT (Seq. Id. No. 7), AAG -GGA-CAA-GCA-GT (Seq. Id. No. 8), CAC-TCC-AGT-CTT-CCA-GT (Seq. Id. No. 9), CAC-TCT -AAG-TCT-CC-AGT (Seq. Id. No. 10), GGA-AAG-CTC-TGT-CTC (Seq. Id. No. 11), GGT-TAC-CCT-ACC-GAC-TT (Seq. Id. No. 12), TAA-AGG-TTA-CCC-TAC-CG (Seq. Id. No. 13), and at least one other of the probes of the probes of the set comprises a probing nucleobase sequence selected from the group consisting of: GCC-ACA-CTT-TAT-CAT-T (Seq. Id. No. 14), GCC-ACA -TCT-TAT-CAT-T (Seq. Id. No. 15), TTC-AAA-AGC-GTG-G (Seq. Id. No. 16), TTC-AAA-GGC-GTG-G (Seq. Id. No. 17), CCT-TTG-TAC-TAT-CCA-TT (Seq. Id. No. 18), GTA-CTA-TCC-AAT-GTA-GC (Seq. Id. No. 19), GAC-CCT-TTG-TAC-TAT-CC (Seq. Id. No. 20), TGG-GAT-TAG-CTC-CAC (Seq. Id. No. 21), GAT-TAG-CTC-CAC-CTC (Seq. Id. No. 22), CTG-AGA-ATA-GTT-TTA-TG (Seq. Id. No. 23), AGA-ATA-GTT-TTA-TGG-GA (Seq. Id. No. 24), ATA-GTT-TTA-TGG-GAT-TAG-C (Seq. Id. No. 25) and TAA-ATT-ATC-TAT-GCT-AA (Seq. Id. No. 26).

In other embodiments, the probe set can comprise two or more independently detectable PNA probes wherein each independently detectable probe is suitable for determining different organisms possibly in a sample and at least one independently detectable probe is suitable for determining organisms of the Listeria genus or for determining organisms of Listeria monocytogenes. Such as assay would be a multiplex assay wherein each of two more bacteria are determined if present in the sample and wherein a suitable independently detectable probe is used for determining each of said bacteria.

c. Methods:

In another embodiment, this invention is directed to methods suitable for determining Listeria bacteria in a sample. Depending upon the nature of the one or more probes used in the method, the method can be used to determine organisms of the Listeria genus or organisms of Listeria monocytogenes. The general and preferred characteristics of PNA probes suitable for determining these bacteria have been previously described herein. Exemplary probing nucleobase sequences are listed in Table 1.

In one embodiment, the method can comprise contacting the sample with one or more PNA probes suitable for determining the Listeria genus, wherein suitable probes have been previously described herein. According to the method, the Listeria in the sample can be determined by correlating hybridization of the probing nucleobase sequence of one or more PNA probes to the target sequence of the bacteria under suitable hybridization conditions or suitable in-situ hybridization conditions. This correlation is made possible by direct or indirect determination of the probe/target sequence complex.

In another embodiment, the method can comprise contacting the sample with one or more PNA probes suitable for determining Listeria monocytogenes, wherein suitable probes have been previously described herein. According to the method, the Listeria monocytogenes in the sample can be determined by correlating hybridization of the probing nucleobase sequence of one or more PNA probes to the target sequence of the bacteria under suitable hybridization conditions or suitable in-situ hybridization conditions. This correlation is made possible by direct or indirect determination of the probe/target sequence complex.

The grouping of PNA probes within probe sets selected for determining certain other bacteria and/or eucarya can also be done. Exemplary probes and probe sets suitable for the practice of this method have been previously described herein. For example, methods for the determination of bacteria, with or without the simultaneous detection of yeast, have been previously described in U.S. Pat. No. 6,280,946, incorporated herein by reference.

Exemplary Assay Formats:

The probes, probe sets, methods and kits of this invention can be used for the detection, identification and/or quantitation of Listeria bacteria. In-situ hybridization (ISH) or fluorescent in-situ hybridization (FISH) can be used as the assay format for detecting, identifying and/or quantitating target organisms. The examples contained herein demonstrate that labeled PNA probes comprising the probing nucleobase sequences listed in Table 1 are reasonably specific for determining target bacteria.

Organisms that have been treated with the PNA probes or probe sets or kits described herein can be determined by several exemplary methods. The cells can be fixed on slides and visualized with a film, camera, slide scanner or microscope. Alternatively, the cells can be fixed and then analyzed in a flow cytometer. Slide scanners and flow cytometers are particularly useful for rapidly quantitating the number of target organisms present in a sample of interest.

d. Kits:

In yet another embodiment, this invention is directed to kits suitable for performing an assay that determines Listeria bacteria in a sample. The general and preferred characteristics of PNA probes suitable for the detection, identification and/or quantitation of Listeria have been previously described herein. Exemplary probing nucleobase sequences are listed in Table 1. Furthermore, methods suitable for using the PNA probes or PNA probe sets of a kit have been previously described herein.

The kits of this invention comprise one or more PNA probes and other reagents or compositions that are selected to perform an assay or otherwise simplify the performance of an assay. The kits can, for example, comprise buffers and/or other reagents useful for performing a PNA-ISH or PNA-FISH assay. In other embodiments, the buffers and/or other reagents can be useful for performing a nucleic acid amplification reaction such as a PCR reaction.

In kits that contain sets of probes, wherein each of at least two probes of the set are used to detect the same or different bacteria or bacteria and yeast. Where two or more different organisms are to be determined, the probes of the set can be labeled with one or more independently detectable moieties so that each specific target organism can be individually determined in a single assay (e.g. a multiplex assay).

e. Exemplary Applications for Using the Invention:

Whether support bound or in solution, the PNA probes, probe sets, methods and kits of this invention can be useful for the rapid, sensitive and reliable detection of Listeria bacteria in food, beverages, water, pharmaceutical products, personal care products, dairy products or for the analysis of environmental samples. The analysis of beverages can include soda, bottled water, fruit juice, beer, wine or liquor products. Suitable PNA probes, probe sets, methods and kits of this invention can be particularly useful for the analysis of raw materials, equipment, products or processes used to manufacture or store food, beverages, water, pharmaceutical products, personal care products, dairy products or for the analysis of environmental samples.

Whether support bound or in solution, the PNA probes, probe sets, methods and kits of this invention are can be useful for the determination of Listeria bacteria in clinical samples and clinical environments. Non-limiting examples of clinical samples include: sputum, laryngeal swabs, gastric lavage, bronchial washings, biopsies, aspirates, expectorates, body fluids (e.g. spinal, pleural, pericardial, synovial, blood, pus, amniotic, and urine), bone marrow and tissue sections. Suitable PNA probes, probe sets, methods and kits can also be particularly useful for the analysis of clinical specimens, equipment, fixtures or products used to treat humans or animals.

### EXAMPLES

This invention is now illustrated by the following examples that are not intended to be limiting in any way.

All PNA oligomers were prepared using conventional synthesis and purification procedures.

### Example 1

### Detection of Listeria Monocytogenes

TABLE 2

List Of PNA Probes Actually Prepared

| Target | |
|---|---|
| organism | Probing Nucleobase Sequence |
| Listeria | Flu-O-AAG-GGA-CAA-GCA-GT-NH₂ |
| species | |
| Listeria | Flu-O-CCC-CAA-CTT-ACA-GGC-NH₂ |
| species | |
| L. | Flu-OO-CCT-TTG-TAC-TAT-CCA-TT-NH₂ |
| monocytogenes | |
| L. | Flu-OO-TGG-GAT-TAG-CTC-CAC-NH₂ |
| monocytogenes | |
| L. | Flu-OO-GAT-TAG-CTC-CAC-CTC-NH₂ |
| monocytogenes | |
| L. | Flu-OO-CTG-AGA-ATA-GTT-TTA-TG-NH₂ |
| monocytogenes | |
| L. | Flu-OO-AGA-ATA-GTT-TTA-TGG-GA-NH₂ |
| monocytogenes | |
| L. | Flu-OO-ATA-GTT-TTA-TGG-GAT-TAG-CG-H₂ |
| monocytogenes | |
| L. | Flu-OO-GGT-TCC-CCC-ATT-CG-NH₂ |
| monocytogenes | |
| L. | Flu-OO-TAC-TTA-TGC-GCC-CTA-NH₂ |
| monocytogenes | |
| L. | Flu-OO-ACG-AAC-CTC-TAA-AGA-NH₂ |
| monocytogenes | |
| Blocker probe | H-CCT-TTG-TAC-CAT-CCA-TT-NH₂ |
| Blocker probe | H-CCT-TTG-TAT-TAT-CCA-TT-NH₂ |
| Blocker probe | H-CTG-AGA-ATG-GTT-TTA-TG-NH₂ |
| Blocker probe | H-CTG-AGA-ATA-ATT-TTA-TG-NH₂ |
| BacUni-1 | Flu-OO-CTG-CCT-CCC-GTA-GGA-NH₂ |

Flu=5(6)-carboxyfluorescein; O=8-amino-3,6-dioxaoctanoic acid

Bacterial Strains

Bacterial strains tested were obtained from The American Type Culture Collection, Manassas, Va. (ATCC), Agricultural Research Service Culture Collection, Peoria, Ill. (NRRL) or University of Wisconsin-Madison, Department of Bacteriology, Stock Culture Collection (DSCC). All bacterial cultures used for this study were grown in Tryptic Soy Broth (Difco laboratories, Detroit, Mich.) at 30° C.

Sample Fixation

A 20 mL aliquot of exponentially growing cultures of Listeria monocytogenes, Listeria innocua, Pseudomonas aeruginosa and Bacillus subtilis were pelleted by centrifugation at 10,000 rpm for 5 minutes, resuspended in 20 mL PBS (7 mM Na₂HPO4; 3 mM NaH2PO4; 130 mM NaCl), pelleted again and resuspended in Fixation Buffer (4% paraformaldehyde in PBS). The bacteria were incubated at room temperature for 60 minutes before they were pelleted again (centrifugation at 10,000 rpm for 5 minutes). After removal of the fixation solution, the cells were resuspended in 20 mL PBS, pelleted and resuspended in 20 mL of 50% aqueous ethanol. The fixed bacteria may be used after 30 minutes of incubation or stored at −20° C. for up to several weeks before being used.

Hybridization

The fixed cells in 50% aqueous ethanol were mixed by vortexing and centrifuged at 10,000 rpm for 5 min. The aqueous ethanol was then removed from the sample and the pellet was resuspended in 100 μL of sterile PBS and pelleted by centrifugation at 10,000 rpm for 5 min. The PBS was removed from the pellet, and the cells were resuspended in 100 μL of hybridization buffer (20 mM Tris-HCl, pH 9.0; 100 mM NaCl; 0.5% SDS) which contained the appropriate probe each at a concentration of 150 pmol/mL. The hybridization was performed at 55° C. for 30 minutes.

The sample was then centrifuged at 10,000 rpm for 5 min. The hybridization buffer was removed and the cells resuspended in 500 μL sterile TE-9.0 (10 mM Tris-HCl, pH 9.0; 1 mM EDTA). The solution was allowed to stand at 55° C. for 10 minutes. The sample was then centrifuged at 10,000 rpm for 5 min. The TE-9.0 was removed from the pellet. This TE-9.0 wash was repeated two more times.

Visualization

After the final wash, the cells were resuspended in 100 μL TE-9.0. An aliquot of 2 μL of this suspension of cells was placed on a glass slide, spread and allowed to dry. Next, 1-2 μL of Vectashield (Vector Laboratories, P/N H-1000) was deposited over the dried cells, a coverslip was added to the slide and its position fixed using a couple of drops of nail polish.

The bacteria were then observed using a Nikon fluorescent microscope equipped with a 60× immersion oil objective, a 10× ocular (total enlargement is 600 fold) and light filters obtained from Omega Optical (XF22 (green), XF34 (red)). Electronic digital images were made of the slide using a SPOT CCD-camera and software obtained from Diagnostic Instruments, Inc., Sterling Heights, Mich. (USA).

Results

The data in Table 3 show that all of the probes tested detected L. monocytogenes. Most showed some degree of cross reactivity with phylogenetically related strains. Future plans include testing of the Listeria genus probes, as well as testing of the L. monocytogenes specific probes with the blocker probe sequences listed above in an effort to improve the specificity of the probes.

TABLE 3

| | | Probes | | | | | |
|---|---|---|---|---|---|---|---|
| Strain | ID | 3 | 4 | 5 | 6 | 9 | 10 |
| L. mono- | FSL-J1 225 | ++ | +++ | +++ | +++ | +++ | ++(+) |
| cytogenes | (Scott A) | | | | | | |
| L. mono- | ATCC | | ++ | ++(+) | | ++(+) | ++(+) |
| cytogenes | #7644 | | | | | | |
| L. innocua | DD680 (M. | +(+) | +/− | − | +/− | ++(+) | ++(+) |
| | Wiedmann) | | | | | | |
| L. innocua | ATCC | | + | +/− | +/− | ++(+) | ++ |
| | #33090 | | | | | | |
| P. aeruginosa | ATCC | | | ++++ | | | |
| | #27853 | | +++ | | | | |
| | | | + | | | | |
| B. subtilis | ATCC | | | | | +/− | ++(+) |
| | #6633 | | | | | | |

### Example 2

### Analysis of Listeria

Cell Growth and Fixation for Hybridization: Cells grown for 18 to 24 hr at 30° C. (or at 25° C. for Brochothrix spp.) in an appropriate medium were harvested by centrifugation (2,000×g, 5 min). All strains except for E. rhusiopathiae, G. haemolysans, C. divergens, C. piscicola and L. fermentum were grown in Columbia broth. E. rhusiopathiae and G. haemolysans were grown in filter-sterilized Columbia broth plus 5% bovine serum (Serum Supreme, Bio Whittaker). The remaining strains were grown in MRS broth. Cells were washed once in PBS, and fixed in either 10% buffered formalin (Sigma) or a 50% solution of absolute ethanol in phosphate buffered saline (PBS). For formalin fixation, washed cells were resuspended in one milliliter of 10% buffered formalin (Sigma) and fixed for one hour at room temperature. Cells were centrifuged (2,000×g, 5 min) and the fixative was removed. Fixed cells were washed again in PBS, resuspended in a 50:50 mixture of absolute ethanol/RNase-free distilled water and stored until use at −20° C. For ethanol-based fixation, cells were harvested as above, washed once, resuspended in 50:50 mixture of absolute ethanol and PBS, then placed for storage at −20° C. As a matter of convenience, most cell preparations were made in advance and stored under these conditions for up to a week prior to hybridization experiments.

Hybridization and Microscopy: Approximately 10⁸ cells (100 μl aliquots of previously prepared cells) were used per hybridization reaction. Cell preparations were centrifuged (2,000×g, 5 min) and the supernatant removed. Cell pellets were resuspended in 50 μl room temperature PNA hybridization buffer (20 mM Tris [pH 9.0], 100 mM NaCl, 0.5% SDS) containing approximately 100 pmol ml⁻¹ of a universal bacterial probe (BacUni-1). Hybridization reactions were performed on a PCR block (DNA Thermal Cycler 480, Perkin Elmer Cetus, Norwalk, Conn.) in 0.5 ml thin-walled PCR tubes (Corning). The PCR machine was programmed to “soak” at 55° C. and timing for each experiment was begun as soon as the PCR block reached the desired hybridization temperature (typically 40-45 s). Cells were hybridized for 1 hr, then 500 μl PNA wash solution (10 mM Tris [pH 9.0], 1 mM EDTA) pre-heated to the hybridization temperature were added to each reaction. Cells were incubated in wash solution for another 10 min, pelleted 2,000×g, 7 min), resuspended in 500 μl fresh, preheated wash solution and incubated for another 20 min at the same temperature. Tubes were thoroughly vortexed whenever PNA wash buffer was added. At the end of this second wash period, hybridized cells were pelleted (2,000×g, 7 min) and resuspended in a small amount (ca. 25-30 μl) of the remaining supernatant. Cell suspensions (2 μl) were smeared onto clean microscope slides (Fisher Scientific) and either air-dried or dried on a PCR block set to 70° C. Bacterial smears were then mounted in VectaShield mounting medium (Vector Labs) and viewed with a fluorescence microscope (Carl Zeiss). Hybridization results were scored as positive (“+”) or negative (“−”). The results of inclusivity and exclusivity properties are summarized in Tables 4 and 5, below.

TABLE 4
Inclusivity Properties of Seq. Id. No. 6 and Seq. Id No. 8 PNA Probes

| | | | Result of Hybridization | | |
|---|---|---|---|---|---|
| Organism | Strain | Notes | BacUni-1 | Seq. Id 6 | Seq. Id 8 |
| Listeria monocytogenes | ATCC 15313 | Type Strain | + | + | + |
| Listeria monocytogenes | FSL-J2-020 | Serotype 1/2a | + | + | + |
| Listeria monocytogenes | FSL-J2-066 | Serotype 1/2a | + | + | + |
| Listeria monocytogenes | FSL-J2-064 | Serotype 1/2b | + | + | + |
| Listeria monocytogenes | FSL-J1-177 | Serotype 1/2b | + | + | + |
| Listeria monocytogenes | FSL-J1-031 | Serotype 4a | + | + | + |
| Listeria monocytogenes | DD6824 | Serotype 4a | + | + | + |
| Listeria monocytogenes | FSL-J1-110 | Serotype 4b | + | + | + |
| Listeria monocytogenes | FSL-C1-122 | Serotype 4b | + | + | + |
| Listeria monocytogenes | DD6821 | Serotype 4c | + | + | + |
| Listeria monocytogenes | ATCC 19118 | Serotype 4e | + | + | + |
| Listeria grayi | KC1773^{A} | Type Strain | + | − | + |
| Listeria grayi | ATCC 25401^{B} | | + | − | + |
| Listeria grayi | ATCC 700545 | | + | − | + |
| Listeria innocua | ATCC 33090 | Type Strain | + | + | + |
| Listeria innocua | ATCC 51742 | | + | + | + |
| Listeria ivanovii | ATCC 19119 | Type Strain | + | + | + |
| Listeria ivanovii | — | | + | + | + |
| Listeria seeligeri | ATCC 35967 | Type Strain | + | + | + |
| Listeria seeligeri | — | | + | + | + |
| Listeria welshimeri | ATCC 35897 | Type Strain | + | + | + |
| Listeria welshimeri | JLJ-20 | | + | + | + |

^{A}Strain is Identical to ATCC 19120
^{B}Type strain for L. murrayi

TABLE 5
Exclusivity Properties of Seq. Id No. 6 and Seq. Id No. 8 PNA Probes

| | | Result of Hybridization | | |
|---|---|---|---|---|
| Organism | Strain | BacUni-1 | Seq. Id 6 | Seq. Id 8 |
| Bacillus cereus | ATCC 11778 | + | − | − |
| Bacillus licheniformis | ATCC 12759 | + | − | − |
| Bacillus subtilis | ATCC 33608 | + | − | − |
| Brochothrix campestris | ATCC 43754 | + | − | − |
| Brochothrix thermosphacta | ATCC 11509 | + | − | − |
| Carnobacterium divergens | NRRL B-14830 | + | − | − |
| Carnobacterium piscicola | NRRL B-14829 | + | − | − |
| Enterococcus faecalis | DSCC 4025 | + | − | − |
| Erysipelothrix rhusiopathiae | ATCC 19414 | + | − | − |
| Gemella haemolysans | ATCC 10379 | + | − | − |
| Kurthia sp. | DSCC 7003 | + | − | − |
| Lactobacillus fermentum | ATCC 14931 | + | − | − |
| Staphylococcus aureus | ATCC 29123 | + | − | − |
| Staphylococcus carnosus | NRRL B-14760 | + | − | − |
| Staphylococcus schleiferi subsp. | NRRL B-14775 | + | − | − |
| schleiferi | | | | |
| Staphylococus xylosus | ATCC 29971 | + | − | − |
| Streptococcus vestibularis | ATCC 49124 | + | − | − |

Having described preferred embodiments of the invention, it will now become apparent to one of skill in the art that other embodiments incorporating the concepts may be used. It is felt, therefore, that these embodiments should not be limited to disclosed embodiments but rather should be limited only by the spirit and scope of the invention.

We claim:

1. A PNA probe comprising a probing nucleobase sequence for detecting, identifying and/or quantitating Listeria in a sample.

2. The PNA probe of claim 1, wherein at least a portion of the probing nucleobase sequence is at least ninety percent homologous to the nucleobase sequences, or their complements, selected from the group consisting of:

| | |
|---|---|
| TTC-CTC-CGT-TCG-TTC-G, | (Seq. Id. No. 1) |
| TAA-GGT-CAT-TCG-TTC-G, | (Seq. Id. No. 2) |
| TTC-GTC-TGT-TCG-TTC-GA, | (Seq. Id. No. 3) |
| AAC-TTT-GGA-AGA-GCA, | (Seq. Id. No. 4) |
| ACG-ACC-AAA-GGA-GC, | (Seq. Id. No. 5) |
| CCC-CAA-CTT-ACA-GGC, | (Seq. Id. No. 6) |
| ACT-CTT-ATC-CTT-GTT-CTT, | (Seq. Id. No. 7) |
| AAG-GGA-CAA-GCA-GT, | (Seq. Id. No. 8) |
| CAC-TCC-AGT-CTT-CCA-GT, | (Seq. Id. No. 9) |
| CAC-TCT-AAG-TCT-CC-AGT, | (Seq. Id. No. 10) |
| GGA-AAG-CTC-TGT-CTC, | (Seq. Id. No. 11) |
| GGT-TAC-CCT-ACC-GAC-TT | (Seq. Id. No. 12) |
| and | |
| TAA-AGG-TTA-CCC-TAC-CG. | (Seq. Id. No. 13) |

3. The PNA probe of claim 1, wherein the probe is unlabeled.

4. The PNA probe of claim 1, wherein the probe is labeled with at least one detectable moiety.

5. The PNA probe of claim 4, wherein the detectable moiety or moieties are selected from the group consisting of: a dextran conjugate, a branched nucleic acid detection system, a chromophore, a fluorophore, a spin label, a radioisotope, an enzyme, a hapten, an acridinium ester and a chemiluminescent compound.

6. The PNA probe of claim 1, wherein the probe is labeled with at least two independently detectable moieties.

7. The PNA probe of claim 6, wherein the two or more independently detectable moieties are independently detectable fluorophores.

8. The PNA probe of claim 1, wherein the probe is support bound.

9. The PNA probe of claim 1, wherein the probe is self-indicating.

10. The PNA probe of claim 1, wherein in situ hybridization is used to determine one or more organisms of Listeria in the sample.

11. A PNA probe comprising a probing nucleobase sequence for detecting, identifying and/or quantitating Listeria monocytogenes in a sample.

12. The PNA probe of claim 11, wherein at least a portion of the probing nucleobase sequence is at least ninety percent homologous to the nucleobase sequences, or their complements, selected from the group consisting of:

| | |
|---|---|
| GCC-ACA-CTT-TAT-CAT-T, | (Seq. Id. No. 14) |
| GCC-ACA-TCT-TAT-CAT-T, | (Seq. Id. No. 15) |
| TTC-AAA-AGC-GTG-G, | (Seq. Id. No. 16) |
| TTC-AAA-GGC-GTG-G, | (Seq. Id. No. 17) |
| CCT-TTG-TAC-TAT-CCA-TT, | (Seq. Id. No. 18) |
| GTA-CTA-TCC-AAT-GTA-GC, | (Seq. Id. No. 19) |
| GAC-CCT-TTG-TAC-TAT-CC, | (Seq. Id. No. 20) |
| TGG-GAT-TAG-CTC-CAC, | (Seq. Id. No. 21) |
| GAT-TAG-CTC-CAC-CTC, | (Seq. Id. No. 22) |
| CTG-AGA-ATA-GTT-TTA-TG, | (Seq. Id. No. 23) |
| AGA-ATA-GTT-TTA-TGG-GA, | (Seq. Id. No. 24) |
| ATA-GTT-TTA-TGG-GAT-TAG-C | (Seq. Id. No. 25) |
| and | |
| TAA-ATT-ATC-TAT-GCT-AA. | (Seq. Id. No. 26) |

13. The PNA probe of claim 11, wherein the probe is unlabeled.

14. The PNA probe of claim 11, wherein the probe is labeled with at least one detectable moiety.

15. The PNA probe of claim 14, wherein the detectable moiety or moieties are selected from the group consisting of: a dextran conjugate, a branched nucleic acid detection system, a chromophore, a fluorophore, a spin label, a radioisotope, an enzyme, a hapten, an acridinium ester and a chemiluminescent compound.

16. The PNA probe of claim 11, wherein the probe is labeled with at least two independently detectable moieties.

17. The PNA probe of claim 16, wherein the two or more independently detectable moieties are independently detectable fluorophores.

18. The PNA probe of claim 11, wherein the probe is support bound.

19. The PNA probe of claim 11, wherein the probe is self-indicating.

20. The PNA probe of claim 11, wherein in situ hybridization is used to determine one or more organisms of Listeria in the sample.

21. A PNA probe set suitable for detecting, identifying and/or quantitating Listeria in a sample.

22. The probe set of claim 11, wherein at least one PNA probe of the set comprises a probing nucleobase sequence wherein at least a portion is at least ninety percent homologous to the nucleobase sequences, or their complements, selected from the group consisting of:

| | |
|---|---|
| TTC-CTC-CGT-TCG-TTC-G, | (Seq. Id. No. 1) |
| TAA-GGT-CAT-TCG-TTC-G, | (Seq. Id. No. 2) |
| TTC-GTC-TGT-TCG-TTC-GA, | (Seq. Id. No. 3) |
| AAC-TTT-GGA-AGA-GCA, | (Seq. Id. No. 4) |
| ACG-ACC-AAA-GGA-GC, | (Seq. Id. No. 5) |
| CCC-CAA-CTT-ACA-GGC, | (Seq. Id. No. 6) |
| ACT-CTT-ATC-CTT-GTT-CTT, | (Seq. Id. No. 7) |
| AAG-GGA-CAA-GCA-GT, | (Seq. Id. No. 8) |
| CAC-TCC-AGT-CTT-CCA-GT, | (Seq. Id. No. 9) |
| CAC-TCT-AAG-TCT-CC-AGT, | (Seq. Id. No. 10) |
| GGA-AAG-CTC-TGT-CTC, | (Seq. Id. No. 11) |
| GGT-TAC-CCT-ACC-GAC-TT, | (Seq. Id. No. 12) |
| TAA-AGG-TTA-CCC-TAC-CG, | (Seq. Id. No. 13) |
| GCC-ACA-CTT-TAT-CAT-T, | (Seq. Id. No. 14) |
| GCC-ACA-TCT-TAT-CAT-T, | (Seq. Id. No. 15) |
| TTC-AAA-AGC-GTG-G, | (Seq. Id. No. 16) |
| TTC-AAA-GGC-GTG-G, | (Seq. Id. No. 17) |
| CCT-TTG-TAC-TAT-CCA-TT, | (Seq. Id. No. 18) |
| GTA-CTA-TCC-AAT-GTA-GC, | (Seq. Id. No. 19) |
| GAC-CCT-TTG-TAC-TAT-CC, | (Seq. Id. No. 20) |
| TGG-GAT-TAG-CTC-CAC, | (Seq. Id. No. 21) |
| GAT-TAG-CTC-CAC-CTC, | (Seq. Id. No. 22) |
| CTG-AGA-ATA-GTT-TTA-TG, | (Seq. Id. No. 23) |
| AGA-ATA-GTT-TTA-TGG-GA, | (Seq. Id. No. 24) |
| ATA-GTT-TTA-TGG-GAT-TAG-C | (Seq. Id. No. 25) |
| and | |
| TAA-ATT-ATC-TAT-GCT-AA. | (Seq. Id. No. 26) |

23. The probe set of claim 21, wherein at least one probe is selected to determine one or more organisms of the Listeria genus while at least one second probe is selected to determine one or more organisms of Listeria monocytogenes.

24. The probe set of claim 23, wherein the at least two probes are independently detectable.

25. The probe set of claim 24, wherein the at least two probes are self-indicating.

26. The probe set of claim 23, wherein a PNA probe for determining organisms of the Listeria genus comprise a probing nucleobase sequence that is at least ninety percent homologous to the nucleobase sequences, or their complements, selected from the group consisting of:

| | |
|---|---|
| TTC-CTC-CGT-TCG-TTC-G, | (Seq. Id. No. 1) |
| TAA-GGT-CAT-TCG-TTC-G, | (Seq. Id. No. 2) |
| TTC-GTC-TGT-TCG-TTC-GA, | (Seq. Id. No. 3) |
| AAC-TTT-GGA-AGA-GCA, | (Seq. Id. No. 4) |
| ACG-ACC-AAA-GGA-GC, | (Seq. Id. No. 5) |
| CCC-CAA-CTT-ACA-GGC, | (Seq. Id. No. 6) |
| ACT-CTT-ATC-CTT-GTT-CTT, | (Seq. Id. No. 7) |
| AAG-GGA-CAA-GCA-GT, | (Seq. Id. No. 8) |
| CAC-TCC-AGT-CTT-CCA-GT, | (Seq. Id. No. 9) |
| CAC-TCT-AAG-TCT-CC-AGT, | (Seq. Id. No. 10) |
| GGA-AAG-CTC-TGT-CTC, | (Seq. Id. No. 11) |
| GGT-TAC-CCT-ACC-GAC-TT, | (Seq. Id. No. 12) |
| TAA-AGG-TTA-CCC-TAC-CG. | (Seq. Id. No. 13) |

27. The probe set of claim 23, wherein a PNA probe for determining organisms of Listeria monocytogenes comprise a probing nucleobase sequence that is at least ninety percent homologous to the nucleobase sequences, or their complements, selected from the group consisting of:

| | |
|---|---|
| GCC-ACA-CTT-TAT-CAT-T, | (Seq. Id. No. 14) |
| GCC-ACA-TCT-TAT-CAT-T, | (Seq. Id. No. 15) |
| TTC-AAA-AGC-GTG-G, | (Seq. Id. No. 16) |
| TTC-AAA-GGC-GTG-G, | (Seq. Id. No. 17) |
| CCT-TTG-TAC-TAT-CCA-TT, | (Seq. Id. No. 18) |
| GTA-CTA-TCC-AAT-GTA-GC, | (Seq. Id. No. 19) |
| GAC-CCT-TTG-TAC-TAT-CC, | (Seq. Id. No. 20) |
| TGG-GAT-TAG-CTC-CAC, | (Seq. Id. No. 21) |
| GAT-TAG-CTC-CAC-CTC, | (Seq. Id. No. 22) |
| CTG-AGA-ATA-GTT-TTA-TG, | (Seq. Id. No. 23) |
| AGA-ATA-GTT-TTA-TGG-GA, | (Seq. Id. No. 24) |
| ATA-GTT-TTA-TGG-GAT-TAG-C | (Seq. Id. No. 25) |
| and | |
| TAA-ATT-ATC-TAT-GCT-AA. | (Seq. Id. No. 26) |

28. The probe set of any of claims 21, 22 or 23, wherein at least one probe of the set is unlabeled.

29. The probe set of any of claims 21, 22 or 23, wherein all probes of the set are unlabeled.

30. The probe set of any of claims 21, 22, 23, 26 or 27, wherein at least one probe is labeled with a detectable moiety.

31. The probe set of any of claims 21, 22, 23, 26 or 27, wherein all probes of the set are labeled with one or more detectable moieties.

32. The probe set of any of claims 30 or 31, wherein the detectable moiety or moieties are selected from the group consisting of: a dextran conjugate, a branched nucleic acid detection system, a chromophore, a fluorophore, a spin label, a radioisotope, an enzyme, a hapten, an acridinium ester and a chemiluminescent compound.

33. The probe set of claim 21, wherein at least one probe of the set is labeled with at least two independently detectable moieties.

34. The probe set of claim 33, wherein the two or more independently detectable moieties are independently detectable fluorophores.

35. The probe set of claim 21, wherein in situ hybridization is used to detect, identify or quantitate one or more organisms in the sample.

36. The probe set of claim 21, wherein two or more probes of the set are independently detectable.

37. The probe set of claim 21, wherein at least one probe of the set is self-indicating.

38. The probe set of claim 21, wherein at least one probe of the set is support bound.

39. The probe set of any of claims 21 or 27, further comprising at least one blocking probe.

40. The probe set of claim 39 wherein one or more of the blocking probes comprise a probing nucleobase sequence that is at least ninety percent homologous to the nucleobase sequences, or their complements, selected from the group consisting of:

| | |
|---|---|
| CCT-TTG-TAC-CAT-CCA-TT, | (Seq. Id. No. 32) |
| CCT-TTG-TAT-TAT-CCA-TT, | (Seq. Id. No. 33) |
| CTG-AGA-ATG-GTT-TTA-TG | (Seq. Id. No. 34) |
| and | |
| CTG-AGA-ATA-ATT-TTA-TG. | (Seq. Id. No. 35) |

41. A method for determining Listeria in a sample; said method comprising:
a) contacting the sample with one or more PNA probes, wherein the one or more PNA probes have a probing nucleobase sequence that is at least ninety percent homologous to the nucleobase sequences, or their complements, selected from the group consisting of:

| | |
|---|---|
| TTC-CTC-CGT-TCG-TTC-G, | (Seq. Id. No. 1) |
| TAA-GGT-CAT-TCG-TTC-G, | (Seq. Id. No. 2) |
| TTC-GTC-TGT-TCG-TTC-GA, | (Seq. Id. No. 3) |
| AAC-TTT-GGA-AGA-GCA, | (Seq. Id. No. 4) |
| ACG-ACC-AAA-GGA-GC, | (Seq. Id. No. 5) |
| CCC-CAA-CTT-ACA-GGC, | (Seq. Id. No. 6) |
| ACT-CTT-ATC-CTT-GTT-CTT, | (Seq. Id. No. 7) |
| AAG-GGA-CAA-GCA-GT, | (Seq. Id. No. 8) |
| CAC-TCC-AGT-CTT-CCA-GT, | (Seq. Id. No. 9) |
| CAC-TCT-AAG-TCT-CC-AGT, | (Seq. Id. No. 10) |
| GGA-AAG-CTC-TGT-CTC, | (Seq. Id. No. 11) |
| GGT-TAC-CCT-ACC-GAC-TT, | (Seq. Id. No. 12) |
| TAA-AGG-TTA-CCC-TAC-CG; | (Seq. Id. No. 13) |
| and | |

b) determining hybridization of the probing nucleobase sequence of a PNA probe to the target sequence in the sample, under suitable hybridization conditions or suitable in-situ hybridization conditions, and correlating the result with the presence, absence or quantity of Listeria in the sample.

42. The method of claim 41, wherein at least one of the PNA probes is unlabeled.

43. The method of claim 41, wherein the one or more PNA probes are all unlabeled.

44. The method of claim 41, wherein at least one PNA probe is labeled with a detectable moiety.

45. The method of claim 41, wherein all probes are labeled with one or more detectable moieties.

46. The method of any of claims 44 or 45, wherein the detectable moiety or moieties are selected from the group consisting of: a dextran conjugate, a branched nucleic acid detection system, a chromophore, a fluorophore, a spin label, a radioisotope, an enzyme, a hapten, an acridinium ester and a chemiluminescent compound.

47. The method of claim 41, wherein at least one PNA probe is labeled with at least two independently detectable moieties.

48. The method of claim 47, wherein the two or more independently detectable moieties are independently detectable fluorophores.

49. The method of claim 41, wherein in situ hybridization using a fluorophore or enzyme-linked probe is used to determine organisms of Listeria in the sample.

50. The method of claim 41, wherein a set of at least two PNA probes is used in the assay.

51. The method of claim 50, wherein two or more PNA probes are independently detectable.

52. The method of claim 41, wherein one or more probes of the set are labeled with two or more independently detectable moieties.

53. The method of claim 52, wherein the two or more independently detectable moieties are independently detectable fluorophores.

54. The method of claim 41, wherein at least one PNA probe is self-indicating.

55. The method of claim 41, wherein at least one PNA probe is support bound.

56. A method for determining Listeria monocytogenes in a sample; said method comprising:
a) contacting the sample with one or more PNA probes, wherein the one or more PNA probes have a probing nucleobase sequence that is at least ninety percent homologous to the nucleobase sequences, or their complements, selected from the group consisting of:

| | |
|---|---|
| GCC-ACA-CTT-TAT-CAT-T, | (Seq. Id. No. 14) |
| GCC-ACA-TCT-TAT-CAT-T, | (Seq. Id. No. 15) |
| TTC-AAA-AGC-GTG-G, | (Seq. Id. No. 16) |
| TTC-AAA-GGC-GTG-G, | (Seq. Id. No. 17) |
| CCT-TTG-TAC-TAT-CCA-TT, | (Seq. Id. No. 18) |
| GTA-CTA-TCC-AAT-GTA-GC, | (Seq. Id. No. 19) |
| GAC-CCT-TTG-TAC-TAT-CC, | (Seq. Id. No. 20) |
| TGG-GAT-TAG-CTC-CAC, | (Seq. Id. No. 21) |
| GAT-TAG-CTC-CAC-CTC, | (Seq. Id. No. 22) |
| CTG-AGA-ATA-GTT-TTA-TG, | (Seq. Id. No. 23) |
| AGA-ATA-GTT-TTA-TGG-GA, | (Seq. Id. No. 24) |
| ATA-GTT-TTA-TGG-GAT-TAG-C | (Seq. Id. No. 25) |
| and | |
| TAA-ATT-ATC-TAT-GCT-AA; | (Seq. Id. No. 26) |
| and | |

b) determining hybridization of the probing nucleobase sequence of a PNA probe to the target sequence in the sample, under suitable hybridization conditions or suitable in-situ hybridization conditions, and correlating the result with the presence, absence or quantity of Listeria monocytogenes in the sample.

57. The method of claim 56, wherein at least one of the PNA probes is unlabeled.

58. The method of claim 56, wherein the one or more PNA probes are all unlabeled.

59. The method of claim 56, wherein at least one PNA probe is labeled with a detectable moiety.

60. The method of claim 56, wherein all probes of the set are labeled with one or more detectable moieties.

61. The method of any of claims 59 or 60, wherein the detectable moiety or moieties are selected from the group consisting of: a dextran conjugate, a branched nucleic acid detection system, a chromophore, a fluorophore, a spin label, a radioisotope, an enzyme, a hapten, an acridinium ester and a chemiluminescent compound.

62. The method of claim 56, wherein at least one PNA probe is labeled with at least two independently detectable moieties.

63. The method of claim 62, wherein the two or more independently detectable moieties are independently detectable fluorophores.

64. The method of claim 56, wherein in situ hybridization using a fluorophore or enzyme-linked probe is used to determine organisms of Listeria in the sample.

65. The method of claim 56, wherein a set of at least two PNA probes is used in the assay.

66. The method of claim 67, wherein the two or more PNA probes are independently detectable.

67. The method of claim 56, wherein one or more probes of the set are labeled with two or more independently detectable moieties.

68. The method of claim 67, wherein the two or more independently detectable moieties are independently detectable fluorophores.

69. The method of claim 56, wherein at least one PNA probe is self-indicating.

70. The method of claim 56, wherein at least one PNA probe is support bound.

71. A kit suitable for performing an assay that determines Listeria in a sample, wherein said kit comprises:
a) one or more PNA probes, wherein the PNA probes comprise a probing nucleobase sequence such that at least a portion is at least ninety percent homologous to the nucleobase sequences, or their complements, selected from the group consisting of:

| | |
|---|---|
| TTC-CTC-CGT-TCG-TTC-G, | (Seq. Id. No. 1) |
| TAA-GGT-CAT-TCG-TTC-G, | (Seq. Id. No. 2) |
| TTC-GTC-TGT-TCG-TTC-GA, | (Seq. Id. No. 3) |
| AAC-TTT-GGA-AGA-GCA, | (Seq. Id. No. 4) |
| ACG-ACC-AAA-GGA-GC, | (Seq. Id. No. 5) |
| CCC-CAA-CTT-ACA-GGC, | (Seq. Id. No. 6) |
| ACT-CTT-ATC-CTT-GTT-CTT, | (Seq. Id. No. 7) |
| AAG-GGA-CAA-GCA-GT, | (Seq. Id. No. 8) |
| CAC-TCC-AGT-CTT-CCA-GT, | (Seq. Id. No. 9) |
| CAC-TCT-AAG-TCT-CC-AGT, | (Seq. Id. No. 10) |
| GGA-AAG-CTC-TGT-CTC, | (Seq. Id. No. 11) |
| GGT-TAC-CCT-ACC-GAC-TT, | (Seq. Id. No. 12) |
| TAA-AGG-TTA-CCC-TAC-CG, | (Seq. Id. No. 13) |
| GCC-ACA-CTT-TAT-CAT-T, | (Seq. Id. No. 14) |
| GCC-ACA-TCT-TAT-CAT-T, | (Seq. Id. No. 15) |
| TTC-AAA-AGC-GTG-G, | (Seq. Id. No. 16) |
| TTC-AAA-GGC-GTG-G, | (Seq. Id. No. 17) |
| CCT-TTG-TAC-TAT-CCA-TT, | (Seq. Id. No. 18) |
| GTA-CTA-TCC-AAT-GTA-GC, | (Seq. Id. No. 19) |
| GAC-CCT-TTG-TAC-TAT-CC, | (Seq. Id. No. 20) |
| TGG-GAT-TAG-CTC-CAC, | (Seq. Id. No. 21) |
| GAT-TAG-CTC-CAC-CTC, | (Seq. Id. No. 22) |
| CTG-AGA-ATA-GTT-TTA-TG, | (Seq. Id. No. 23) |
| AGA-ATA-GTT-TTA-TGG-GA, | (Seq. Id. No. 24) |
| ATA-GTT-TTA-TGG-GAT-TAG-C | (Seq. Id. No. 25) |
| and | |
| TAA-ATT-ATC-TAT-GCT-AA; | (Seq. Id. No. 26) |
| and | |

b) other reagents or compositions necessary to perform the assay.

72. The kit of claim 71, wherein the probes of the kit are unlabeled.

73. The kit of claim 71, wherein at least one probe is labeled with a detectable moiety.

74. The kit of claim 71, wherein two or more probes are labeled with independently detectable moieties.

75. The kit of claim 74, wherein the independently detectable moieties are independently detectable fluorophores.

76. The kit of claim 71, wherein at least one probe is labeled with at least two independently detectable moieties.

77. The kit of claim 76, wherein the two or more independently detectable moieties are independently detectable fluorophores.

78. The kit of claim 72, wherein hybridization of the probing nucleobase sequence of the probe to the nucleic acid of the organism of interest is detected using an antibody or antibody fragment, wherein the antibody or antibody fragment specifically binds to the PNA/nucleic acid complex.

79. The kit of claim 79, further comprising an antibody labeled with a detectable moiety.

80. The kit of claim 79, wherein the detectable moiety is selected from the group consisting of a dextran conjugate, a branched nucleic acid detection system, a chromophore, a fluorophore, a spin label, a radioisotope, an enzyme, a hapten, an acridinium ester and a chemiluminescent compound.

81. The kit of claim 71, further comprising buffers and/or other reagents suitable for performing a PNA-ISH or PNA-FISH assay.

80. The kit of claim 71, further comprising buffers and/or other reagents suitable for performing a nucleic acid amplification reaction.

81. The kit of claim 71, wherein at least one PNA probe is self-indicating.

82. The kit of claim 71, wherein the kit comprises at least one blocking probe.

83. The kit of claim 82, wherein one or more of the blocking probes comprise a probing nucleobase sequence that is at least ninety percent homologous to the nucleobase sequences, or their complements, selected from the group consisting of:

| | |
|---|---|
| CCT-TTG-TAC-CAT-CCA-TT, | (Seq. Id. No. 32) |
| CCT-TTG-TAT-TAT-CCA-TT, | (Seq. Id. No. 33) |
| CTG-AGA-ATG-GTT-TTA-TG, | (Seq. Id. No. 34) |
| and | |
| CTG-AGA-ATA-ATT-TTA-TG. | (Seq. Id. No. 35) |
